(19) United States
(12) Patent Application Publication
(10) Pub. No.: US 2015/0283149 A1
(43) Pub. Date: Oct. 8, 2015

(54) METHODS OF TREATING PATIENTS HAVING IMPLANTABLE CARDIAC DEVICES

(71) Applicant: Gilead Sciences, Inc., Foster City, CA (US)

(72) Inventors: Luiz Belardinelli, Palo Alto, CA (US); Ewa Prokopczuk, Danville, CA (US); Dewan Zeng, Palo Alto, CA (US)

(21) Appl. No.: 14/664,177

(22) Filed: Mar. 20, 2015

Related U.S. Application Data
(60) Provisional application No. 61/981,483, filed on Apr. 18, 2014.
(60) Provisional application No. 61/974,384, filed on Apr. 2, 2014.

(51) Int. Cl. A61K 31/553

(52) U.S. Cl. CPC A61K 31/553

(57) ABSTRACT

Described herein are methods for reducing ventricular tachycardia/ventricular fibrillation (VT/VF), methods for reducing implantable cardioverter-defibrillator device (ICD) shocks, methods for reducing CV hospitalization, ER visits, CV death or SCD in patients having implantable cardiac devices or CRT, and methods for reducing the use of ICD in a human patient without prior VT/VF, by administering an effective amount of the potent and selective late sodium ion channel blocker Compound 1.

### CROSS REFERENCE TO RELATED APPLICATIONS

This application claims benefit under 35 U.S.C. §119(e) to U.S. Provisional Patent Application Ser. No. 61/974,384, filed on Apr. 2, 2014 and U.S. Provisional Patent Application Ser. No. 61/981,483, filed on Apr. 18, 2014, the entireties of which are incorporated herein by reference.

### FIELD

The present disclosure relates to methods of treating patients having implantable cardiac devices by administration of an effective amount of a potent and selective late sodium ion channel blocker.

### BACKGROUND

Ventricular tachycardia (VT) and ventricular fibrillation (VF) are the major causes of sudden cardiac death (SCD). Currently many patients with a history of or at risk for VT/VF are treated with cardiac rhythm management devices such as implantable cardioverter-defibrillator devices (ICD). (See, for example, Russo A M, Stainback R F, Bailey S R, Epstein A E, Heidenreich P A, Jessup M, et al. ACCF/HRS/AHA/ASE/HFSA/SCAI/SCCT/SCMR 2013 Appropriate Use Criteria for Implantable Cardioverter-Defibrillators and Cardiac Resynchronization Therapy: a Report of the American College of Cardiology Foundation Appropriate Use Criteria Task Force, Heart Rhythm Society, American Heart Association, American Society of Echocardiography, Heart Failure Society of America, Society for Cardiovascular Angiography and Interventions, Society of Cardiovascular Computed Tomography, and Society for Cardiovascular Magnetic Resonance. Heart Rhythm 2013;10 (4):e11-58.)

ICDs have been shown to prevent SCD by terminating the episodes of VT/VF. They are used as a standard of care for secondary prevention SCD in patients with a history of VT/VF. There is also a growing use of ICDs for primary prevention in patients at high risk of developing VT/VF, typically those with left ventricular (LV) dysfunction post myocardial infarction, low LV ejection fraction (LVEF), and who may have other risk factors. Heart failure patients meeting specific criteria are treated with cardiac rhythm management devices such as cardiac resynchronization therapy-defibrillator (CRT-D) devices that combine cardiac resynchronization therapy using bi-ventricular pacing with ICD therapy.

ICDs terminate ventricular arrhythmias, but do not prevent them. Nearly 50% of patients with ICD will require adjuvant antiarrhythmic drug (AAD) therapy to reduce the frequency of VT/VF and subsequently ICD shocks. (See, for example, Williams E S, Viswanathan M N. Current and Emerging Antiarrhythimc Drug Therapy for Ventricular Tachycardia. Cardiology and Therapy 2013;2 (1):27-46.) Shocks are physically painful and psychologically traumatic for the patient and frequent shocks are associated with higher mortality and increased health care expenditures (e.g., hospitalizations). (See, for example, Larsen G K, Evans J, Lambert W E, Chen Y, Raitt M H. Shocks Burden and Increased Mortality in Implantable Cardioverter-Defibrillator Patients. Heart Rhythm 2011;8 (12):1881-6 and Van Herendael H, Pinter A, Ahmad K, Korley V, Mangat I, Dorian P. Role of Antiarrhythmic Drugs in Patients with Implantable Cardioverter Defibrillators. Europace: European Pacing, Arrhythmias, and Cardiac Electrophysiology: Journal of the Working Groups on Cardiac Pacing, Arrhythmias, and Cardiac Cellular Electrophysiology of the European Society of Cardiology 2010;12 (5):618-25.)

Despite the unmet medical need for adjuvant AAD therapy in patients with ICDs, there are no currently approved medications indicated for the reduction of ICD shocks and/or reduction of CV hospitalization or CV death including SCD. The most commonly used agent, amiodarone, has a poor benefit-risk profile, including significant organ-toxicity and has recently been shown to increase mortality in patients with ICD (ALPHEE study). (See, for example, Kowey P R, Crijns H J, Aliot E M, Capucci A, Kulakowski P, Radzik D, et al. Efficacy and Safety of Celivarone, with Amiodarone as Calibrator, in Patients with an Implantable Cardioverter-Defibrillator for Prevention of Implantable Cardioverter-Defibrillator Interventions or Death: the ALPHEE study. Circulation 2011;124 (24):2649-60.) The other AADs (such as sotalol or dofetilide) used in patients with ICD are associated with pro-arrhythmic risk. (See, for example, Droogan C, Patel C, Yan G X, Kowey P R. Role of Antiarrhythmic Drugs: Frequent Implantable Cardioverter-Defibrillator Shocks, Risk of Proarrhythmia, and New Drug Therapy. Heart Failure Clinics 2011;7 (2):195-205, viii and Reiffel J A. Adjunctive Therapy for Recurrent Ventricular Tachycardia in Patients with Implantable Cardioverter Defibrillators. Current Cardiology Reports 2007;9 (5):381-6.)

Thus, there is a need to provide methods for reducing ventricular tachycardia/ventricular fibrillation (VT/VF). Also there is a need to provide methods of reducing the medical interventions, such as antitachycardia pacing (ATP), defibrillation shock, or the like, in patients having implantable cardiac rhythm management devices. Also, there is a need to provide methods for reducing CV hospitalization, ER visits, CV death or SCD in in patients having implantable cardiac rhythm management devices. Also, there is a need to provide methods for reducing the use of or the need for ICD in patients without prior VT/VF.

### SUMMARY

4-(Pyrimidin-2-ylmethyl)-7-(4-(trifluoromethoxy)phenyl)-3,4-dihydrobenzo[f][1,4]oxazepin-5(2H)-one or Compound 1 is a potent and selective inhibitor of the cardiac late sodium current (Na). See, U.S. Pub. No. 2013/0012492. It is contemplated that Compound 1 is effective in reducing medical interventions in a human patient having an implantable cardiac rhythm management device. It is also contemplated that Compound 1 is effective in reducing ventricular tachycardia/ventricular fibrillation (VT/VF) in a human patient having an implantable cardiac rhythm management device.

Provided herein is a method for reducing medical interventions in a human patient having an implantable cardiac rhythm management device, said method comprising administering to the human patient an effective amount of Compound 1:

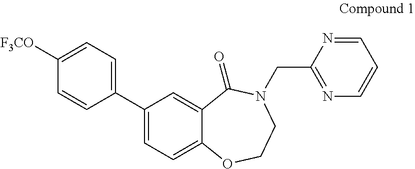 [SMILES: O=C1c2cc(-c3ccc(OC(F)(F)F)cc3)ccc2OCCN1Cc1ncccn1]

or a pharmaceutically acceptable salt thereof.

Also provided herein is a method for reducing ventricular tachycardia/ventricular fibrillation (VT/VF) in a human patient having an implantable cardiac rhythm management device, said method comprising administering to the human patient an effective amount of Compound 1:

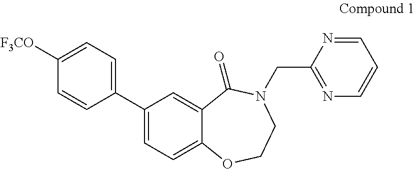 [SMILES: O=C1c2cc(-c3ccc(OC(F)(F)F)cc3)ccc2OCCN1Cc1ncccn1]

or a pharmaceutically acceptable salt thereof.

Also provided herein is a method for reducing sudden cardiac death (SCD) in a human patient having an implantable cardiac rhythm management device, said method comprising administering to the human patient an effective amount of Compound 1:

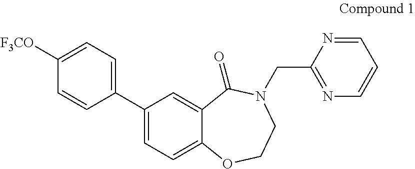 [SMILES: O=C1c2cc(-c3ccc(OC(F)(F)F)cc3)ccc2OCCN1Cc1ncccn1]

or a pharmaceutically acceptable salt thereof.

Also provided herein is a method for reducing ICD shocks in a human patient having an ICD, said method comprising administering to the human patient an effective amount of Compound 1:

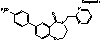 [SMILES: O=C1c2cc(-c3ccc(OC(F)(F)F)cc3)ccc2OCCN1Cc1ncccn1]

or a pharmaceutically acceptable salt thereof.

Also provided herein is a method for reducing the use of ICD in a human patient having an ICD, said method comprising administering to the human patient an effective amount of Compound 1:

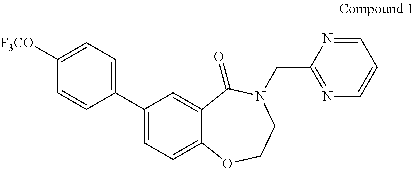 [SMILES: O=C1c2cc(-c3ccc(OC(F)(F)F)cc3)ccc2OCCN1Cc1ncccn1]

or a pharmaceutically acceptable salt thereof.

Also provided herein is a method for reducing the need for implantable cardioverter-defibrillator device (ICD) in a human patient having an ICD, said method comprising administering to the human patient an effective amount of Compound 1:

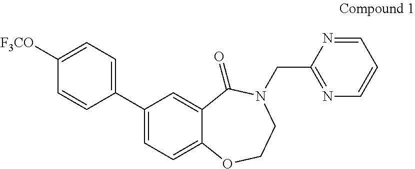 [SMILES: O=C1c2cc(-c3ccc(OC(F)(F)F)cc3)ccc2OCCN1Cc1ncccn1]

or a pharmaceutically acceptable salt thereof.

### DETAILED DESCRIPTION

### 1. Definitions

As used in the present specification, the following words and phrases are generally intended to have the meanings as set forth below, except to the extent that the context in which they are used indicates otherwise.

It is to be noted that as used herein and in the claims, the singular forms “a,” “an” and “the” include plural referents unless the context clearly dictates otherwise. Thus, for example, reference to “a pharmaceutically acceptable carrier” in a composition includes two or more pharmaceutically acceptable carriers, and so forth.

“Comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure. Embodiments defined by each of these transition terms are within the scope of this disclosure.

The term “effective amount” refers to that amount of a compound that is sufficient to effect treatment, as defined below, when administered to a mammal in need of such treatment. The effective amount will vary depending upon the specific activity of the therapeutic agent being used, the severity of the patient's disease state, and the age, physical condition, existence of other disease states, and nutritional status of the patient. Additionally, other medication the patient may be receiving will affect the determination of the effective amount of the therapeutic agent to administer.

The term “treatment” or “treating” means administration of Compound 1 to a mammal, particularly a human patient in need thereof, for purposes including: 1) preventing or protecting against the disease or condition, that is, causing the clinical symptoms not to develop; 2) inhibiting the disease or condition, that is, arresting or suppressing the development of clinical symptoms; and/or 3) relieving the disease or condition, as disclosed herein, that is causing the regression of clinical symptoms. In some embodiments, the term “treatment” or “treating” refers to relieving the disease or condition that is causing the regression of clinical symptoms.

As used herein, the term “preventing” refers to the prophylactic treatment of a patient in need thereof. The prophylactic treatment can be accomplished by providing an appropriate dose of Compound 1 to a subject at risk of suffering from an ailment, thereby substantially averting onset of the ailment.

It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the tetra “prophylaxis” is intended as an element of “treatment” to encompass both “preventing” and “suppressing” as defined herein. The term “protection,” as used herein, is meant to include “prophylaxis.”

The term “patient” typically refers to a “mammal” which includes, without limitation, human, monkeys, rabbits, mice, domestic animals, such as dogs and cats, farm animals, such as cows, horses, or pigs, and laboratory animals. In some embodiments, the term patient refers to a human in need of treatment as defined herein.

“Administering” or “administration” refers to the delivery of compound 1 and/or more therapeutic agents to a patient. In one embodiment, the administration is coadministration such that two or more therapeutic agents are delivered together at one time. In certain embodiments, two or more therapeutic agents can be coformulated into a single dosage form or “combined dosage unit”, or formulated separately and subsequently combined into a combined dosage unit, typically for intravenous administration or oral administration.

“Intravenous administration” is the administration of substances directly into a vein, or “intravenously”. Compared with other routes of administration, the intravenous (IV) route is the fastest way to deliver fluids and medications throughout the body. An infusion pump can allow precise control over the flow rate and total amount delivered, but in cases where a change in the flow rate would not have serious consequences, or if pumps are not available, the drip is often left to flow simply by placing the bag above the level of the patient and using the clamp to regulate the rate. Alternatively, a rapid infuser can be used if the patient requires a high flow rate and the IV access device is of a large enough diameter to accommodate it. This is either an inflatable cuff placed around the fluid bag to force the fluid into the patient or a similar electrical device that may also heat the fluid being infused. When a patient requires medications only at certain times, intermittent infusion is used, which does not require additional fluid. It can use the same techniques as an intravenous drip (pump or gravity drip), but after the complete dose of medication has been given, the tubing is disconnected from the IV access device. Some medications are also given by IV push or bolus, meaning that a syringe is connected to the IV access device and the medication is injected directly (slowly, if it might irritate the vein or cause a too-rapid effect). Once a medicine has been injected into the fluid stream of the IV tubing there must be some means of ensuring that it gets from the tubing to the patient. Usually this is accomplished by allowing the fluid stream to flow normally and thereby carry the medicine into the bloodstream; however, a second fluid injection is sometimes used, a “flush”, following the injection to push the medicine into the bloodstream more quickly.

“Oral administration” is a route of administration where a substance is taken through the mouth, and includes buccal, sublabial and sublingual administration, as well as enteral administration and that through the respiratory tract, unless made through e.g. tubing so the medication is not in direct contact with any of the oral mucosa. Typical form for the oral administration of therapeutic agents includes the use of tablets or capsules.

A “sustained release formulation” is a formulation which is designed to slowly release a therapeutic agent in the body over an extended period of time, whereas an “immediate release formulation” is a formulation which is designed to quickly release a therapeutic agent in the body over a shortened period of time. In some cases the immediate release formulation may be coated such that the therapeutic agent is only released once it reached the desired target in the body (e.g. the stomach).

The formulae of Compound 1 is also intended to represent unlabeled forms as well as isotopically labeled forms. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive

The term “pharmaceutically acceptable salt” of a given compound refers to salts that retain the biological effectiveness and properties of the given compound, and which are not biologically or otherwise undesirable. Pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl)amines, tri(substituted alkyl)amines, alkenyl amines, dialkenyl amines, trialkenyl amines, substituted alkenyl amines, di(substituted alkenyl)amines, tri(substituted alkenyl)amines, cycloalkyl amines, di(cycloalkyl)amines, tri(cycloalkyl)amines, substituted cycloalkyl amines, disubstituted cycloalkyl amine, trisubstituted cycloalkyl amines, cycloalkenyl amines, di(cycloalkenyl)amines, tri(cycloalkenyl)amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Amines are of general structure)N(R³⁰)(R³¹)(R³²), wherein mono-substituted amines have two of the three substituents on nitrogen (R³⁰ R³¹, and R³²) as hydrogen, di-substituted amines have one of the three substituents on nitrogen (R³⁰, R³¹, and R³²) as hydrogen, whereas tri-substituted amines have none of the three substituents on nitrogen (R³⁰, R³¹, and R³²) as hydrogen. R³⁰, R³¹, and R³² are selected from a variety of substituents such as hydrogen, optionally substituted alkyl, aryl, heteroaryl, cycloalkyl, cycloalkenyl, heterocyclyl, and the like. The above-mentioned amines refer to the compounds wherein either one, two, or three substituents on the nitrogen are as listed in the name. For example, the term “cycloalkenyl amine” refers to cycloalkenyl-NH₂, wherein “cycloalkenyl” is as defined herein. The term “diheteroarylamine” refers to NH(heteroaryl)₂, wherein “heteroaryl” is as defined herein, and so on.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, or unless otherwise indicated herein, its use in the therapeutic compositions is contemplated. Supplementary active ingredients can also be incorporated into the compositions.

The term “implantable cardiac rhythm management device” refers to a device implantable in a patient to treat irregular or other abnormal cardiac rhythms by delivering electrical pulses to the patient's heart. Implantable cardiac rhythm management devices include, among other things, pacemakers, also referred to as pacers. Pacers are often used to treat patients with bradyarrhythmias, that is, hearts that beat too slowly. Such pacers may also coordinate atrial and ventricular contractions to improve the heart's pumping efficiency. Implantable cardiac rhythm management devices also include devices providing cardiac resynchronization therapy (CRT), such as for patients with congestive heart failure (CHF). CHF patients have dysfunctional heart muscles that display reduced contractility and cause poorly synchronized heart contraction patterns. By pacing multiple heart chambers in a coordinated fashion, the CRT device restores a more synchronized contraction of the weakened heart muscle, thus increasing the heart's efficiency as a pump. Implantable cardiac rhythm management devices also include defibrillators that are capable of delivering higher energy electrical stimuli to the heart. Such defibrillators may also include cardioverters, which synchronize the delivery of such stimuli to portions of sensed intrinsic heart activity signals. Defibrillators are used to treat patients with tachyarrhythmias, that is, hearts that beat too quickly. In addition to pacers, CRT devices, and defibrillators, implantable cardiac rhythm management devices also include, among other things, pacer/defibrillator devices that combine the functions of pacers and defibrillators, drug delivery devices, and any other implantable systems or devices for diagnosing or treating cardiac arrhythmias. One of skill in the art is aware of the placement of implantable devices for the management of cardiac rhythm

The term “medical intervention” refers to interventions to manage cardiac rhythm in patients. Such interventions include, but are not limited to drug therapy, pacing defibrillation, cardioversion, and other intervention techniques known in the art.

The phrase “reducing the need for implantable cardiac rhythm management device (ICD)” means that a qualified physician or care giver can based on the patient's history or presenting data decide to substitute therapy by providing an ICD placement with therapy based at least in part on treatment with the compound of formula (I). The phrase also means that therapy with Compound 1 or the ICD supplements each other acting as a backup therapy mode, one for the other, such that the overall need for activation of the ICD is reduced or diminished. It is contemplated that in some patients treatment with compound 1 will fundamentally change the role of an ICD and in some cases remove the need for an ICD as determined by the qualified care giver.

The phrase “reducing ventricular tachycardia/ventricular fibrillation (VT/VF)” includes terminating occurrences or recurrences of VT/VF, reducing recurrence frequency of VT/VF, preventing occurrences of new VT/VF episodes.

The phrase “reducing sudden cardiac death (SCD)” means reducing the likelihood of sudden cardiac death in a patient having an ICD based on the additional beneficial effect afforded by administration of Compound 1.

The phrase “reducing ICD shocks” means reducing the frequency of firing (shocking) by an ICD based on the beneficial effect of Compound 1 to terminate or prevent VT/VF thereby reducing the need for or frequency of ICD shocks being administered by the ICD device.

Likelihood of sudden cardiac death in a patient having an ICD based on the additional beneficial effect afforded by administration of Compound 1.

### 2. Methods

Generally, the present disclosure relates to a method for reducing medical interventions in a human patient having an implantable cardiac rhythm management device, said method comprising administering to the human patient an effective amount of Compound 1:

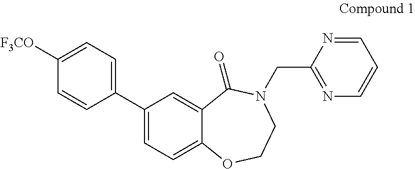 [SMILES: O=C1c2cc(-c3ccc(OC(F)(F)F)cc3)ccc2OCCN1Cc1ncccn1]

or a pharmaceutically acceptable salt thereof.

In one embodiment, Compound 1 is administered intravenously. In another embodiment, Compound 1 is administered orally. In another embodiment, the effective amount of Compound 1 is from about 1 mg to about 300 mg per day. In another embodiment, the effective amount of Compound 1 is about 3 mg, 6 mg, 10 mg, 20 mg, 30 mg, 40 mg, 50 mg, 60 mg or 100 mg per day. In another embodiment, Compound 1 is administered once a day. In another embodiment, the implantable cardiac rhythm management device is a pacer or a defibrillator. In another embodiment, the implantable cardiac rhythm management device is a defibrillator. In another embodiment, the implantable cardiac rhythm management device is an implantable cardioverter defibrillator (ICD) or a cardiac resynchronization therapy defibrillator (CRT-D). In another embodiment, the medical interventions comprise an antitachycardia pacing therapy or a defibrillation shock.

The present disclosure also relates to a method for reducing ventricular tachycardia/ventricular fibrillation (VT/VF) in a human patient having an implantable cardiac rhythm management device, said method comprising administering to the human patient an effective amount of Compound 1:

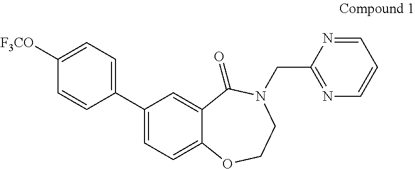 [SMILES: O=C1c2cc(-c3ccc(OC(F)(F)F)cc3)ccc2OCCN1Cc1ncccn1]

or a pharmaceutically acceptable salt thereof.

The present disclosure also relates to a method for reducing sudden cardiac death (SCD) in a human patient having an implantable cardiac rhythm management device, said method comprising administering to the human patient an effective amount of Compound 1:

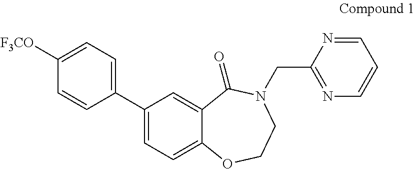 [SMILES: O=C1c2cc(-c3ccc(OC(F)(F)F)cc3)ccc2OCCN1Cc1ncccn1]

or a pharmaceutically acceptable salt thereof.

In one embodiment, Compound 1 is administered intravenously. In another embodiment, Compound 1 is administered orally. In another embodiment, the effective amount of Compound 1 is from about 1 mg to about 300 mg per day. In another embodiment, the effective amount of Compound 1 is about 3 mg, 6 mg, 10 mg, 20 mg, 30 mg, 40 mg, 50 mg, or 60 mg per day. In another embodiment, Compound 1 is administered once a day. In another embodiment, the implantable cardiac rhythm management device is a pacer or a defibrillator. In another embodiment, the implantable cardiac rhythm management device is a defibrillator. In another embodiment, the implantable cardiac rhythm management device is an implantable cardioverter defibrillator (ICD) or a cardiac resynchronization therapy defibrillator (CRT-D).

The present disclosure also relates to a method for reducing ICD shocks in a human patient having an ICD, said method comprising administering to the human patient an effective amount of Compound 1:

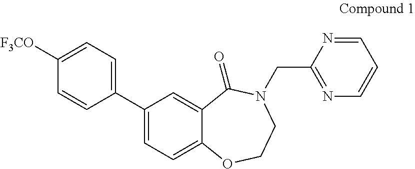 [SMILES: O=C1c2cc(-c3ccc(OC(F)(F)F)cc3)ccc2OCCN1Cc1ncccn1]

The present disclosure also relates to a method for reducing the use of implantable cardioverter-defibrillator device (ICD) in a human patient having an ICD, said method comprising administering to the human patient an effective amount of Compound 1:

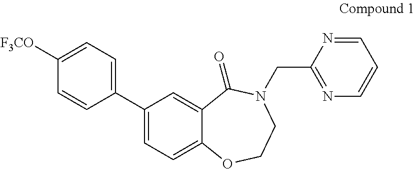 [SMILES: O=C1c2cc(-c3ccc(OC(F)(F)F)cc3)ccc2OCCN1Cc1ncccn1]

or a pharmaceutically acceptable salt thereof.

The present disclosure also relates to a method for reducing the need for implantable cardioverter-defibrillator device (ICD) in a human patient having an ICD, said method comprising administering to the human patient an effective amount of Compound 1:

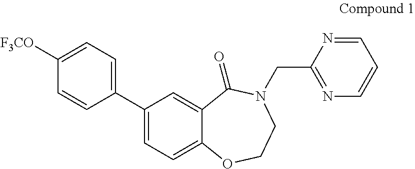 [SMILES: O=C1c2cc(-c3ccc(OC(F)(F)F)cc3)ccc2OCCN1Cc1ncccn1]

or a pharmaceutically acceptable salt thereof.

In one embodiment, Compound 1 is administered intravenously. In another embodiment, Compound 1 is administered orally. In another embodiment, the effective amount of Compound 1 is from about 1 mg to about 300 mg per day. In another embodiment, the effective amount of Compound 1 is about 3 mg, 6 mg, 10 mg, 20 mg, 30 mg, 40 mg, 50 mg, or 60 mg per day. In another embodiment, Compound 1 is administered once a day.

In one embodiment, this disclosure provides methods as described herein wherein the cardiac rhythm management device is not implantable.

In another embodiment, this disclosure provides methods to reduce the overall occurrence of appropriate ICD interventions (ATP or shock); methods to reduce premature ventricular complex (PVC); methods to reduce nonsustained ventricular tachycardia (nsVT) episodes; methods to reduce the overall occurrence of VT; methods to reduce ICD interventions (ATP or shock) or cardiovascular (CV) death; methods to reduce the overall occurrence of electrical storm; methods to reduce CV hospitalization, emergency room (ER) visit, or CV death in subjects with ICD or CRT-D.

### 3. Compound

Embodiments of the present disclosure comprise Compound 1:

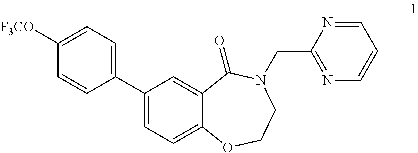 [SMILES: O=C1c2cc(-c3ccc(OC(F)(F)F)cc3)ccc2OCCN1Cc1ncccn1]

which is named 4-(pyrimidin-2-ylmethyl)-7-(4-(trifluoromethoxy)phenyl)-3,4-dihydrobenzo[f][1,4]oxazepin-5(2H)-one, or a pharmaceutically acceptable salt thereof.

Compound 1 may be prepared as described in U.S. Pub. No. 2013/0012492, which is hereby incorporated by reference for all purposes in its entirety. Compound 1 is a potent late sodium channel inhibitor (see, U.S. Pub. No. 2013/0012492).

### 4. Dosing

Compound 1 may be administered to the patient in either single or multiple doses by any of the accepted modes of administration of agents having similar utilities, for example as described in those patents and patent applications incorporated by reference, including buccal, by intra-arterial injection, intravenously, intraperitoneally, parenterally, intramuscularly, subcutaneously, orally, or via an impregnated or coated device such as a stent, for example, or an artery-inserted cylindrical polymer. In one embodiment, Compound 1 is administered intravenously.

In one embodiment, Compound 1 is administered orally, such as, for example, in a tablet. In one embodiment, Compound 1 is administered to a human patient in need thereof in an effective amount, such as, e.g., from about 1 mg to about 1 g per day. In one embodiment, the effective amount is from about 1 mg to about 300 mg per day. In other embodiments, the effective amount is about 3 mg, about 6 mg, about 10 mg, about 20 mg, about 30 mg, about 40 mg, about 50 mg, about 60 mg, about 80 mg, or about 100 mg per day. In certain embodiments, the compound is administered once a day. In practicing the method of the present disclosure, one of skill in the art is able to, based on, the patient's history and/or presentation administer the Compound 1 as an adjunct to a cardiac rhythm management system, e.g. an ICD. A qualified care giver may dose the Compound 1 according to objective requirements for further management of the patient's disease condition in view of the presence of an ICD. A qualified care giver may remove an ICD or reduce the frequency of shocks provided by an ICD or take other measures leveraging the beneficial effects of Compound 1 in a patient having or contemplated to have an ICD.

### 5. Pharmaceutical Formulations

Formulations contemplated by the present disclosure may also be for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present disclosure. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the component in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In making pharmaceutical compositions that include Compound 1, the active ingredient is usually diluted by an excipient or carrier and/or enclosed within such a carrier that can be in the form of a capsule, sachet, paper or other container. When the excipient serves as a diluent, it can be a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compounds, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The compositions of the disclosure may be formulated so as to provide quick, sustained or delayed release of the active ingredient after administration to the patient by employing procedures known in the art. In certain embodiments, sustained release formulations are used. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345.

The compositions are preferably formulated in a unit dosage form. The term “unit dosage forms” or “combined dosage unit” refers to physically discrete units suitable as unitary dosages for human subjects or other mammals, each unit containing a predetermined quantity of the active materials calculated to produce the desired effect, in association with a suitable pharmaceutical excipient (e.g., a tablet, capsule, ampoule). The active agents of the disclosure are effective over a wide dosage range and are generally administered in an effective amount. It will be understood, however, that the amount of each active agent actually administered will be determined by a physician, in the light of the relevant circumstances, including the condition to be treated, the chosen route of administration, the actual compounds administered and their relative activity, the age, weight, and response of the individual patient, the severity of the patient's symptoms, and the like.

For preparing solid compositions such as tablets, the principal active ingredients are mixed with a pharmaceutical excipient to form a solid preformulation composition containing a homogeneous mixture of a compound of the present disclosure. When referring to these preformulation compositions as homogeneous, it is meant that the active ingredients are dispersed evenly throughout the composition so that the composition may be readily subdivided into equally effective unit dosage forms such as tablets, pills and capsules.

The tablets or pills of the present disclosure may be coated or otherwise compounded to provide a dosage form affording the advantage of prolonged action, or to protect from the acid conditions of the stomach. For example, the tablet or pill can comprise an inner dosage and an outer dosage element, the latter being in the form of an envelope over the former. Compound 1 and the co-administered agent(s) can be separated by an enteric layer that serves to resist disintegration in the stomach and permit the inner element to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Activity testing is conducted in the Examples below using methods described herein and those well known in the art.

### EXAMPLES

The following example is included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### List of Abbreviations and Acronyms.

| Abbreviation | Meaning |
|---|---|
| g | Gram |
| mg | Milligram |

### Example 1

### Study to Evaluate Effect of Compound 1 on Ventricular Arrhythmia in Patients with Implantable Cardiac Rhythm Management Devices

A randomized, double-blind, placebo-controlled, dose ranging, parallel group study is designed to assess the effects of Compound 1 (3 mg, 6 mg and 30 mg) compared with placebo in patients with an implantable cardioverter defibrillator (ICD) or a cardiac resynchronization therapy defibrillator (CRT-D).

Approximately 210 patients are divided into different groups in a 1:1:1 ratio to receive either Compound 1 in 2 different doses or matching placebo for at least 24 weeks. Randomization is stratified by implanted device (ICD or CRT-D) and by region United States vs Rest of World.

One group of patients receives a single loading dose of 30 mg of Compound 1 on day 1, followed by 6 mg once daily of Compound 1 for 6 months or up to approximately 18 months. Another group of patients receives a single loading dose of 30 mg of Compound 1 on day 1, followed by 3 mg daily of Compound 1 for 6 months or up to approximately 18 months. Another group(s) of patients receive a single loading dose of 30 mg of placebo on day 1, followed by 6 mg of placebo once daily for 6 months or up to approximately 18 months. Compound 1 as well as the placebo is administered orally in tablet form.

Patients who qualified for study entry, based on the inclusion criteria, are randomized to a treatment sequence. The main inclusion criteria is patients having an ICD or CRT-D implanted for primary or secondary prevention and at least one ICD intervention for VT/VF (shock or ATP) within 60 days prior to screening or a documented VT/VF episode (prior to implantation) within 60 days prior to screening. Patients are seen in the clinic at screening, randomization (Day 1 of Treatment period), Week 4, Week 12, Week 24, and every 12 weeks thereafter until the last patient randomized is followed up for approximately 24 weeks. A safety follow-up contact occurs 30 (+5) days after the last dose of study drug.

Safety is assessed by collecting adverse events (AEs), clinical laboratory tests, vital signs, PE findings, and electrocardiogram (ECG) data. Physical examination (PE) findings before the first dose of the study drug will be captured as medical history and post dose events will be AEs.

The primary efficacy objective is to evaluate the effect of Compound 1 compared to placebo on the overall occurrence of appropriate ICD interventions (shock or ATP) in patients with ICD or CRT-D during the first 24 weeks of treatment.

The secondary efficacy objectives of the study are to evaluate the effect of Compound 1 compared to placebo on the following: the overall occurrence of appropriate ICD interventions (ATP or shock) in subjects with ICD or CRT-D through the end of the study; premature ventricular complex (PVC) count/24 hours after 12 weeks of treatment (cECG monitoring); nonsustained ventricular tachycardia (nsVT) episodes/24 hours after 12 weeks of treatment (cECG monitoring); the overall occurrence of VT (treated and untreated) during the first 24 weeks and through the end of the study; the time from randomization to the first occurrence of appropriate ICD interventions (ATP or shock) or cardiovascular (CV) death; the overall occurrence of electrical storm during the first 24 weeks and through the end of the study; electrical storm is defined as ≧3 separate episodes of ventricular arrhythmia within a 24 hour period terminated by ICD; the occurrence of inappropriate ICD interventions during the first 24 weeks and through the end of the study; time from randomization to the first CV hospitalization, emergency room (ER) visit, or CV death; left ventricular (LV) systolic and diastolic function as assessed by echocardiography (ECHO); and safety and tolerability change in non-sustained ventricular tachycardia (nsVT).

We claim:

1. A method for reducing medical interventions in a human patient having an implantable cardiac rhythm management device, said method comprising administering to the human patient an effective amount of Compound 1: 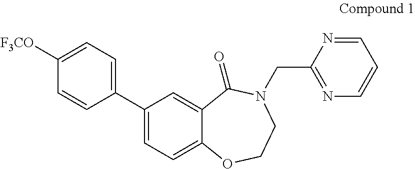 [SMILES: O=C1c2cc(-c3ccc(OC(F)(F)F)cc3)ccc2OCCN1Cc1ncccn1]
or a pharmaceutically acceptable salt thereof.

2. A method for reducing ventricular tachycardia/ventricular fibrillation (VT/VF) in a human patient having an implantable cardiac rhythm management device, said method comprising administering to the human patient an effective amount of Compound 1: 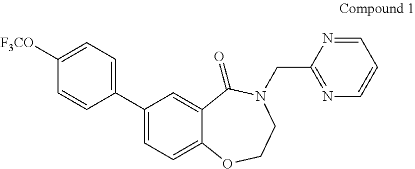 [SMILES: O=C1c2cc(-c3ccc(OC(F)(F)F)cc3)ccc2OCCN1Cc1ncccn1]
or a pharmaceutically acceptable salt thereof.

3. A method for reducing sudden cardiac death (SCD) in a human patient having an implantable cardiac rhythm management device, said method comprising administering to the human patient an effective amount of Compound 1: 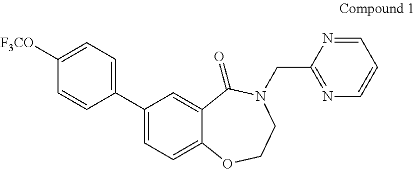 [SMILES: O=C1c2cc(-c3ccc(OC(F)(F)F)cc3)ccc2OCCN1Cc1ncccn1]
or a pharmaceutically acceptable salt thereof.

4. A method for reducing implantable cardioverter-defibrillator device (ICD) shocks in a human patient having an ICD, said method comprising administering to the human patient an effective amount of Compound 1: 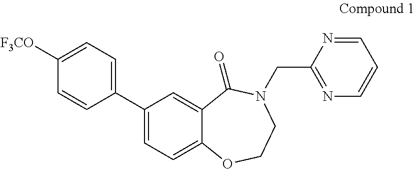 [SMILES: O=C1c2cc(-c3ccc(OC(F)(F)F)cc3)ccc2OCCN1Cc1ncccn1]
or a pharmaceutically acceptable salt thereof.

5. A method for reducing the need for implantable cardioverter-defibrillator device (ICD) in a human patient having an ICD, said method comprising administering to the human patient an effective amount of Compound 1: 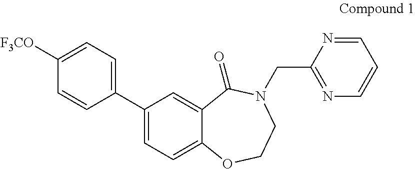 [SMILES: O=C1c2cc(-c3ccc(OC(F)(F)F)cc3)ccc2OCCN1Cc1ncccn1]
or a pharmaceutically acceptable salt thereof.

6. The method of claim 1, wherein Compound 1 is administered intravenously.

7. The method of claim 1, wherein Compound 1 is administered orally.

8. The method of claim 1, wherein the effective amount of Compound 1 is from about 1 mg to about 300 mg per day.

9. The method of claim 1, wherein the effective amount of Compound 1 is about 3 mg, 6 mg, 10 mg, 20 mg, 30 mg, 40 mg, 50 mg, or 60 mg per day.

10. The method of claim 1, wherein Compound 1 is administered once a day.

11. The method of claim 1, wherein the implantable cardiac rhythm management device is a pacer or a defibrillator.

12. The method of claim 1, wherein the medical interventions comprises an antitachycardia pacing therapy or a defibrillation shock.

13. The method of claim 2, wherein Compound 1 is administered intravenously.

14. The method of claim 2, wherein Compound 1 is administered orally.

15. The method of claim 2, wherein the effective amount of Compound 1 is from about 1 mg to about 300 mg per day.

16. The method of claim 2, wherein the effective amount of Compound 1 is about 3 mg, 6 mg, 10 mg, 20 mg, 30 mg, 40 mg, 50 mg, or 60 mg per day.

17. The method of claim 2, wherein Compound 1 is administered once a day.

18. The method of claim 2, wherein the implantable cardiac rhythm management device is a pacer or a defibrillator.

19. The method of claim 3, wherein Compound 1 is administered intravenously.

20. The method of claim 3, wherein Compound 1 is administered orally.

21. The method of claim 3, wherein the effective amount of Compound 1 is from about 1 mg to about 300 mg per day.

22. The method of claim 3, wherein the effective amount of Compound 1 is about 3 mg, 6 mg, 10 mg, 20 mg, 30 mg, 40 mg, 50 mg, or 60 mg per day.

23. The method of claim 3, wherein Compound 1 is administered once a day.

24. The method of claim 3, wherein the implantable cardiac rhythm management device is a pacer or a defibrillator.

25. The method of claim 4, wherein Compound 1 is administered intravenously.

26. The method of claim 4, wherein Compound 1 is administered orally.

27. The method of claim 4, wherein the effective amount of Compound 1 is from about 1 mg to about 300 mg per day.

28. The method of claim 4, wherein the effective amount of Compound 1 is about 3 mg, 6 mg, 10 mg, 20 mg, 30 mg, 40 mg, 50 mg, or 60 mg per day.

29. The method of claim 4, wherein Compound 1 is administered once a day.

30. The method of claim 5, wherein Compound 1 is administered intravenously.

31. The method of claim 5, wherein Compound 1 is administered orally.

32. The method of claim 5, wherein the effective amount of Compound 1 is from about 1 mg to about 300 mg per day.

33. The method of claim 5, wherein the effective amount of Compound 1 is about 3 mg, 6 mg, 10 mg, 20 mg, 30 mg, 40 mg, 50 mg, or 60 mg per day.

34. The method of claim 5, wherein Compound 1 is administered once a day.
